(19) United States
(12) Patent Application Publication
(10) Pub. No.: US 2008/0183200 A1
(43) Pub. Date: Jul. 31, 2008

(54) METHOD OF SELECTIVE AND CONTAINED ULTRASOUND DEBRIDEMENT

(75) Inventor: Eilaz Babaev, Minnetonka, MN (US)

Correspondence Address: Bacoustics, LLC, 5929 BAKER ROAD, SUITE 470, MINNETONKA, MN, 55345, US

(73) Assignee: BACOUSTICS LLC, Minnetonka, MN (US)

(21) Appl. No.: 12/060,450

(22) Filed: Apr. 1, 2008

Related U.S. Application Data
(63) Continuation-in-part of application No. 11/857,162, filed on Sep. 18, 2007.
Application No. 11/857,162 is a continuation-in-part of application No. 11/449,220, filed on Jun. 7, 2006.

(51) Int. Cl. A61B 17/32; A61N 7/00

(52) U.S. Cl. 606/169; 601/2

(57) ABSTRACT

A method of ultrasound assisted wound debridement is disclosed. The method comprises dissecting material to be debrided with an ultrasonically vibrating cutting structure. Splatter created during the procedure is captured with ultrasound waves directed towards a collection point. Capturing splatter, the delivered ultrasound waves reduce and/or eliminate the amount splattered escaping into ambient air. The material to be debrided is dissected with an ultrasonically vibrating cutting that induces vibrations within the material to be debrided. The induced vibrations carry energy into the material that strains the adhesion holding the material together and/or to the wound, selectively separating susceptible materials while potentially sparing healthy, healing, and/or granulation tissue from debridement.

### CROSS REFERENCE TO RELATED APPLICATIONS

This application is a continuation-in-part of U.S. patent application Ser. No. 11,857,162 filed Sep. 18, 2007, the teachings of which are hereby incorporated by reference.

This application is also a continuation-in-part of U.S. patent application Ser. No. 11/449,220 filed Jun. 7, 2006, the teachings of which are hereby incorporated by reference.

### BACKGROUND OF THE INVENTION

1. Field of the Invention

The present invention relates to a method of debriding wounds and/or dissecting tissues from the body.

2. Description of the Related Art

When confronted with wounded tissue, physicians and similar practitioners of the medical arts may rely on numerous methods to treat the wound. Despite the repertoire of available techniques, treating severe and/or chronic wounds can be especially difficult. To assist in healing, the practitioner may have to resort to surgical debridement. During debridement, the practitioner removes material from a wound to expose healthy and/or granulation tissue. It is generally believed that keeping such tissue exposed expedites wound healing. However, as wounds begin to heal after debridment, additional material may collect over the healthy and/or granulation tissue. Successful treatment, therefore, often requires repeated debridement.

A diverse amount of material may be removed from the wound during debridment. The removed material may include improperly healed, dead, and/or dying tissue. However, the material removed is not limited to tissue. For instance, foreign substances such as, but not limited to, dirt, debris, and/or infectious agents may collect within the wound. In the case of an infectious agent such as, but not limited to, a bacteria, a bacterial laden biofilm may develop over the wound covering healthy and/or granulation tissue. As the infection increases in severity, the wound may become covered with gangrenous tissue. Exposing healthy and/or granulation tissue would then require the removal of such contaminants. Accordingly, foreign contaminants, biofilms, and/or gangrenous tissue may represent material that has to be debrided from the wound.

In combination or in the alternative, the trauma and/or pathological conditions creating the wound may generate material that needs to be removed to expose healthy and/or granulation tissue. The trauma responsible for creating the wound may fracture blood vessels supplying tissue surviving the trauma. Fracturing the blood vessels, the trauma reduces the blood supply to the surviving tissue creating a region of ischemic tissue. Ischemia may also be the result of various conditions such as, but not limited, diabetes and/or various vascular diseases. As the ischemia persists, the tissue becomes deprived of vital nutrients required for growth and/or survival, and thus may eventually become devitalized. Failing to receive required nutrients, the devitalized tissue may eventually slip into a non-viable state. The non-viable tissue may begin a process of necrosis and/or apoptosis in which the cells of the non-viable tissue release various factors that digest and/or degrade the tissue. Destroying itself, the non-viable tissue becomes necrotic tissue. If the degradation and/or digestive process continues beyond the point of cellular death, the necrotic tissue may become slough. However, it is also possible that digestion and/or degradation stops with cellular death as to create an eschar over the wound. Regardless of how far the tissue progresses from ischemia and/or devitalization to slough and/or eschar, the dead and dying tissue generated from trauma and/or pathological condition responsible for the wound should be removed.

In combination or in the alternative, the wounds may also generate material that needs to be removed to expose healthy and/or granulation tissue. For instance, in response to an inflammation brought about by the presence of foreign substances and/or trauma an exudate may be secreted. As the secreted exudate persists, the wound may become covered by various proteins and/or other molecules manufactured by the body. Secretion of a fibrinous exudate, for example, may lead a buildup of fibrin over the wound. Regardless of the type of exudate secreted and/or built-up over the wound, this body-generated material should be removed during debridement.

In combination or in the alternative, improperly healed tissue may have to be removed from the wound. For example, instead of migrating into the wound, cells responsible for closing the wound and/or replacing lost tissue may begin to migrate away from the wound. Such misdirected migration may hinder and/or prevent proper closure and/or healing of the wound. Placing cellular migration on the proper path may require removing the tissue created as a result of the misdirected migration. Otherwise healthy tissue hindering, preventing, or otherwise hindering proper closure and/or healing of the wound may appear through other means. Regardless of its origin and/or condition, tissue hindering closure and/or healing should be removed during debridement.

Debriding a wound to expose healthy and/or granulation tissue and/or remove tissue hindering proper closure and/or healing is generally done to expedite wound healing. Exposing debrided wounds to ultrasound has also been shown to expedite wound healing. As to capitalize on this phenomenon, various debridement techniques with ultrasonically vibrating instruments have been developed. Typically these techniques entail removing various materials from the wound with ultrasonically vibrating dissection devices, while simultaneously exposing the wound to ultrasound. Accordingly the wound receives beneficial ultrasound while it is debrided.

### SUMMARY OF THE INVENTION

Debriding a wound to expose healthy and/or granulation tissue and/or to remove material hindering proper closure and/or healing is generally done to free the wound of various and/or potential burdens hindering healing. Debriding a wound with an ultrasonically vibrating dissection device has the added benefit of exposing the wound to beneficial ultrasound. However, the benefits derived from ultrasound debridement can become outweighed by its burdensome nature. For instance, indiscriminate dissection resulting in the removal of healthy, healed, and/or granulation tissue can burden the patient by creating a deeper wound and/or requiring re-healing properly healed tissue. Additionally, inducing motion with the material removed, the ultrasonic vibrations of a dissection device may splatter the removed material. Splattering the removed material may lead to a contamination of the surrounding environment, burdening other patients within the clinic. Other patients exposed to the contaminated environment may develop a wide range of complications such as, but not limited to infection. Restoring a safe sterility may require an extensive cleaning. Patients requiring access to the environment to receive needed therapies will have to wait until the cleaning process has been completed. Consequently, ultrasound assisted wound debridement, despite its potential benefits, is a burdensome procedure.

As to lessen and/or eliminate these burdens, a method of ultrasound assisted wound debridement is disclosed. The method comprises removing, fragmenting, and/or otherwise dissecting material to be debrided with an ultrasonically vibrating cutting structure. Splatter created during the procedure is captured with ultrasound waves directed towards a collection point. The collection point may be any surface upon which the splatter can accumulate such as, but not limited to, a collection device and/or the patient's own body. The ultrasound waves capture the splatter by forcing it towards the collection point. Capturing splatter, the delivered ultrasound waves reduce and/or eliminate the amount splattered escaping into ambient air. Consequently, the present invention reduces and/or eliminates burdensome contamination.

The present invention also reduces and/or eliminates the burdensome removal of healthy, healing, and/or granulation tissue. Dissecting material to be debrided with an ultrasonically vibrating cutting structure induces vibrations within the material to be debrided and/or spared. The induced vibrations carry energy into the material that strains the adhesion holding the material together and/or to the wound. Adhesion susceptible to this strain is broken causing certain materials to be separated from the wound and/or fragmented. Material able to withstand the strain such as healthy, healing, and/or granulation tissue will remain intact and in place. Consequently, the induced vibrations selectively separate susceptible materials while sparing healthy, healing, and/or granulation tissue from debridement. Selectively sparing healthy, healing, and/or granulation tissue, the present inventions reduces and/or eliminates unintentional deepening and/or re-injuring of the wound. Accordingly, the present invention lessens and/or eliminates burdens associated with ultrasound assisted wound debridement.

Indiscriminate dissection during debridement burdens the patients by removing healthy, healing, and/or granulation tissue. Deepening the injury and/or re-injuring the wound, the removal of such tissue burdens the patient by setting back healing. Healthy tissue and/or tissue spared from the trauma and/or pathology resulting in the wound does not need to be healed and/or repaired. Having escaped injury, this tissue can be thought of as marking the boundaries of the wound. Accordingly, removing and/or injuring this tissue during debridement would deepen and/or expand the wound. Expanding the wound results in more tissue that has to be replaced and/or healed. Consequently, the indiscriminate dissection of healthy tissue during debridement burdens the patient by creating more to heal.

In combination or in the alternative to creating more to heal, indiscriminate dissection may burden the patient by removing healing and/or healed tissue. As the wound heals, injured tissue may be repaired and/or lost tissue and/or irreparably-injured tissue replaced. Recovering from the trauma and/or pathology resulting in the wound and/or replacing lost and/or irreparably injured tissue, healed and/or healing tissue marks the boundaries of the healing. Removing and/or injuring this tissue during debridement would, consequently, push back the boundaries of wound healing because the removed and/or injured tissue would have to be re-healed to restore the original boundaries of healing. Consequently, the indiscriminate dissection of healing and/or healed tissue may, burden the patient by necessitating re-healing.

In combination or in the alternative, a repeated effort may also result from the indiscriminate dissection of granulation tissue during debridement. In the early stages of healing, granulation tissue supporting tissue repair and/or replacement fills the wound. As granulation tissue fills the wound it forms a provisional extracellular matrix. New cells replacing lost tissue may utilize the provisional extracellular matrix to migrate into the wound, and eventually anchor themselves to it. Consequently, the provisional extracellular matrix formed by granulation tissue can be thought of as scaffolding upon which new tissue is built. In addition to providing a framework for new tissue, granulation tissue also comprises newly formed blood vessels supplying nutrients needed to repair and/or replace lost and/or injured tissue. Granulation tissue, therefore, provides the foundations of healing. Indiscriminately dissecting this tissue during debridement may require these foundations to be rebuilt before healing could progress. Consequently, the indiscriminate dissection of granulation tissue may burden the patient by necessitating a rebuilding of the foundations of healing.

Re-injuring and/or deepening the wound, indiscriminate debridement burdens the patient. Of course, the goal of debridement is not to burden the patient. Rather, it is the goal of debridement to improve healing by removing material covering healthy and/or granulation tissue and/or hindering proper closure and/or healing. Typically the material is removed with an indiscriminate cutting device such as scalpel. Recently, various ultrasonic dissection devices have been developed for debridement. As these devices remove tissue indiscriminately, debridement techniques employing them rely on the practitioner to differentiate between material to be debrided and that to be retained. Unfortunately, it is often difficult for the medical practitioner performing debridment to differentiate between the material to be debrided and that to be retained. This may be because the material to be debrided generally covers the wound, making it difficult for the practitioner to know how deep to debride. In combination or in the alternative, it may be hard to differentiate between the material to be debrided and that to be retained. Consequently, the practitioner may remove the good with the bad.

Inducing vibrations within the tissue and/or other material about the point of dissection with an ultrasonically vibrating cutting structure may allow for susceptibility-selective separation. As the cutting device advances through the wound, vibrations are induced within the tissue and/or other material about the point of dissection. The induced vibrations cause the material to compress and rebound, inducing motion within the cells and/or molecules within the material. As the cells and/or molecules move, they gain kinetic energy due to their motion. Inducing vibrations, therefore, adds energy to the material about the point of dissection that is carried by the induced vibrations.

The energy carried by the induced vibrations can be utilized to break the adhesion holding the material to be debrided together and/or to the wound. Tissue and other materials are held together and/or to the wound by various adhesions. For instance, the associations between proteins may form at least a portion of the adhesion. In combination or in the alternative, adhesion may be formed from the associations between the various elements forming the material. Regardless of their specific nature, a large portion of the adhesion holding material together and/or to the wound may be chemical interactions breakable by an input of energy. The amount of energy required to break chemical interactions is variable. Each interaction requires a specific energy to break it. However, only a set amount of energy is carried into the material about the point of dissection by inducing vibrations with an ultrasonically vibrating cutting structure. Of this energy, only a portion of it will be absorbed by the adhesion holding material together and/or to the wound. Consequently, as the ultrasound vibrating cutting structure advances through the wound, a set amount of energy will be absorbed by the adhesion holding material within the wound together and/or to the wound. Depending on the material, the amount of energy absorbed may be sufficient or insufficient to break adhesion holding the material together and/or to the wound. Accordingly, the material within the wound will have differing susceptibility to the energy carried by the vibrations induced by the advancement of the ultrasonically vibrating cutting structure, allowing for susceptibility selective separation.

Healthy tissue is held in place by cellular adhesion capable of withstanding the energy carried by the vibrations induced about the point of dissection. Cellular adhesion is created from the expression of various molecules such as, but not limited to, cellular adhesion molecules. These proteins on the cell surface generally provide adhesion by binding to proteins on the surface of neighboring cells and/or to the extracellular matrix. Typically, these proteins anchor themselves to the cell's cytoskeleton and extend past the cellular membrane to bind to the cell's surroundings. Binding to the cell's surroundings and/or anchoring themselves to the cytoskeleton, cellular adhesion molecules provide an adhesion that can withstand energy carried by the induced vibrations.

Expressing cellular adhesion as it grows into the wound, granulation tissue and/or other developing tissue becomes increasingly resistant to the energy carried by the induced vibrations. Initially, the synthesized cellular adhesion assists the newly formed tissue to find its proper place. After the tissue and/or its constituent cells have migrated to their proper place, the tissue grows and/or matures, synthesizing new cellular adhesion. As the prevalence of the new and/or mature cellular adhesion increases, the tissue becomes more firmly rooted in place. Increasing its adhesion with new and/or maturing cellular adhesion, granulation tissue and/or other developing tissue become increasingly resistant to the energy carried by the induced vibrations.

Just as growing and/or developing tissue is marked by an increasing resistance to the energy carried by the induced vibrations, dying tissue is marked by an increasing susceptibility. As the cells of devitalized, ischemic, and/or infected tissue begin to die, the prevalence of cellular adhesion begins to decrease. The declining prevalence of cellular adhesion may result from a down regulation of its expression, as certain cellular adhesion has to be regularly regenerated. In other words, the adhesion eventually fails and has to be replaced. However, when cells begin to experience apoptosis and/or necrosis they stop repairing and/or replacing adhesion molecules. No longer repairing and/or replacing failing adhesion molecules, dead and/or dying cells lose their adhesive hold. Furthermore, as necrosis and/or apoptosis progresses enzymes are released that digest and/or degrade the tissue and/or its components. Consequently, the tissue loses its adhesion to itself and/or its surroundings. Accordingly, the adhesion holding dead, dying, and/or devitalized tissue together and/or to the wound become increasingly weakened. Losing its adhesion, dead, dying, and/or devitalized tissue becomes increasingly susceptible to the energy carried by the induced vibrations.

Granulation, devitalized, dead, and/or dying tissue are not the only materials within a wound experiencing a changing susceptibility to the energy carried by the induced vibrations. As the wound heals, proteins and/or other material secreted by the body may experience decreasing adhesion to the wound.

In an effort to protect healthy and/or growing tissue, the body may secrete various materials that bind to the wound to form a hardened protective barrier. This protective barrier prevents foreign material, such as, but not limited to, dirt, debris, and/or infectious agents from entering the wound. However, the secreted material may also hinder growing granulation tissue and/or migrating epidermal cells from entering the wound. Migrating epidermal cells such as, but not limited to, keratinocytes typically digest and/or degrade the barrier, separating it from the wound. Consequently, as the wound heals the adhesion holding the barrier to the wound weakens, making the barrier increasingly susceptible to the energy carried by the induced vibrations.

In addition to losing the adhesion holding them to the wound, secreted materials may also lose the adhesion holding them together. For instance, moistening the material may weaken adhesion holding the material together. Water, saline, and/or other fluids utilized to moisten the material may disrupt the associations formed between the secreted materials. Disrupting adhesive associations, the fluid moistening the secreted materials soften the barrier they form making it more susceptible to fragmentation by the energy carried by the induced vibrations.

Material secreted by the body may not form a hardened barrier. Instead the secreted material may resemble fluid exudates, gel like substances, and/or other soft substances. The weak adhesion between the material forming soft substances covering the wound makes them susceptible to fragmentation by the energy carried by the induced vibrations.

Weak adhesion may also be found between infectious agents and the wound. Infectious agents may express various surface molecules that provide adhesion to the wound. As with cellular adhesion molecules, molecules expressed by infectious agents may bind to the extracellular matrix and/or cells of various tissues. However, unlike cellular adhesion molecules, adhesion molecules expressed by infectious agents are generally not adapted to specifically bind to the extracellular matrix and/or cellular adhesion molecules expressed within the tissue. Accordingly, the adhesion displayed is the result of an inexact fit between molecules expressed by the infectious agent and tissues of the body. Failing to fit exactly, the binding of the infectious agent's surface molecules creates a weaker adhesion more susceptible to the energy carried by the induced vibrations.

Not all foreign material entering the wound forms appreciable adhesion. Consequently, certain foreign material such as, but not limited to, dirt, debris, and/or infectious agents lacking appropriate surface molecules may simply rest on the surface of the wound. Failing to establish appreciable adhesion, such foreign material is highly susceptible to the energy carried by the induced vibrations.

Vibrations are induced within material about the point of dissection by the advancement of an ultrasonically vibrating cutting structure. Any structure containing an ultrasonically vibrating edge and/or point sufficiently fine as to enable dissection may be utilized as the vibrating cutting structure. For instance, an instrument with an ultrasonically vibrating sharpened structure may be utilized as the vibrating cutting structure. The sharpened structure could be any element such as an edge and/or point that has been sharpened to some degree. Though sharpened to some degree, the sharpened structure does not have to have an edge and/or point so fine as to be capable of easily performing dissection in the absence of ultrasonic vibrations. For instance, a structure with an edge and/or point to blunt to allow for easy dissection may be utilized. The ability to use such a blunt cutting structure to dissect the material to be debrided is due to energy carried by the vibrations induced and transferred to the material about the point of dissection.

Transferring the energy they carry to the material within the wound, the induced vibrations selectively separate susceptible adhesions holding material together and/or the wound. Vibrations are induced in the material about the point of dissection by transferring the vibrations from the cutting structure to the material. However, it is possible that the material within the wound may dampen the vibrations of the cutting structure. Advancing the cutting structure through the material within the wound may cause the material to come into direct contact with the structure's edge and/or point. This will add mass to the cutting structure, thereby dampening its vibrations. Dampening the vibrations may lessen the energy transferred to the material within the wound, possibly sparing otherwise susceptible adhesion. It may, therefore, be beneficial to isolate the cutting structure from the material within the wound.

Isolating the cutting structure from the material within the wound can be accomplished by moistening the wound. The wound may be moistened with a variety of fluids, such as saline, water, vegetable oil, alcohol, and/or any other fluid not unduly toxic to the wound. As the cutting structure advances through a moistened wound, the fluid utilized to moisten the wound flows between the cutting structure and the material within the wound preventing direct contact between the structure and the material. This allows the cutting structure to vibrate more freely. In addition to isolating the cutting structure from the material within the wound, the fluid moistening the wound may act as a conduit carrying vibrations from the cutting structure to and/or into the material about the point of dissection.

Not only do vibrations induce motion within material by causing it to compress and rebound, they are also transmitted through a material by the induced motion. Inducing a motion within a material requires that the molecules and/or various matter making up the material are capable of moving. The ability of molecules and/or other matter making up a material to move is influenced by, among other things, the freedom of movement allowed by the adhesion between the molecules and/or other matter of the material. For instance, molecules and/or other matter held together by an adhesion not allowing movement about and/or along the adhesion may not compress forward when struck by an advancing vibration. Failing to move forward, it cannot induce molecules and/or matter in front of it to move forward, which may block the further transmission of the vibration. Blocking the transmission of vibrations, adhesion may prevent the energy carried by the vibrations from traveling deeper into the tissue. Instead of traveling forward, a large portion of the energy carried by the blocked vibration may be absorbed by the adhesion and/or surrounding material.

Adhesion allowing movement about and/or along the adhesion as to allow for further transmission of the vibrations may also absorb energy carried by the vibration. As vibrations travel through materials, a portion of the energy they carry is constantly being transferred and lost every time one molecule and/or matter induces another to move. Inducing molecules and/or matter to move requires energy. A portion of the energy used to induce one molecule and/or matter to move is transferred to the molecules and/or matter it induces to move. Consequently, as vibrations travel through a material at least a portion of the energy carried is used to transmit the vibrations. However, every time energy is transferred a portion of it is lost. Some of the lost energy may be absorbed by the adhesion holding the material together and/or to the wound. Consequently, as the vibrations are transmitted through a material the adhesion holding the material together and/or to the wound may absorb a portion of the energy the vibrations carry. If the adhesion absorbs a sufficient amount of energy the material it holds may become separated from itself and/or the wound.

It is possible that the material about the point of dissection may not allow for the transmission of vibrations. This may be overcome by moistening the wound. The fluid utilized to moisten the wound may permeate into the material. Once within the material, it may act as a conduit for ultrasound vibrations. Accordingly, the vibrating cutting structure may induce vibrations within the fluid that are transmitted through the fluid by the movement of the molecules and/or matter comprising the fluid. Traveling through the fluid permeating the material within the wound, the vibrations may be conducted by the fluid through the material. Accordingly, fluid utilized to moisten the wound may serve as conduit for carrying vibrations to, into and/or through material. Acting as conduit carrying vibrations, the fluid may increase the amount of energy transferred to the material within the wound.

Traveling away from the vibrating cutting structure and into the wound, the induced vibrations carry energy into the material about the point of dissection. Because the vibrations are induced within material in front of the cutting structure, the energy carried by the vibrations travels ahead of the vibrating cutting structure. A portion of energy traveling through the material within the wound may be absorbed by the adhesion holding the material together and/or to the wound. If the energy absorbed is insufficient to break the adhesion, the material will remain intact and/or in place. If, however, the energy absorbed equals or exceeds the energy required to break the adhesion, the material will be separated from itself and/or the wound. Consequently, the energy carried by the induced vibrations traveling ahead of the cutting structure will begin breaking and/or weakening the adhesion holding susceptible material together and/or to the wound before the cutting structure reaches the material. Selectively separated ahead of the cutting device, the material to be debrided can be lifted away, pushed aside, fragmented, and/or otherwise dissected by the advancing cutting device.

The energy selectively separating susceptible material will also be absorbed by the material debrided from the wound. Accordingly, the material debrided from the wound will have an energy to it. Because the energy imparted on the debrided material removed from the wound is carried by ultrasonic vibrations induced within the material, the energy is at least in part kinetic energy. As the material separates from the wound and/or itself, the kinetic energy will be transferred to motion. This transfer of energy to motion may splatter the material into the ambient air.

Escaping into the ambient air, the splattered debrided material may contaminate other patients and/or the facility, thereby burdening the patient population and/or clinic. Capturing at least a portion of the splatter may eliminate and/or lessen this burden. At least a portion of the splatter may be captured with ultrasound waves having trajectories directed towards a collection point. The trajectory of an ultrasound wave is the direction the wave travels away from its point of release. As a wave travels it compresses, or forces things forward, about its trajectory. As such, an ultrasound wave exerts a force about its trajectory. Consequently, when splatter crosses the path of an ultrasound wave having a trajectory directed towards a collection point a force directed towards the collection is exerted on the splatter. Exerting a force on the splatter, the delivered ultrasound wave accelerates the splatter's velocity towards the collection point, and possibly decelerating its velocity towards the ambient. Accelerating splatter's velocity towards a collection point, the ultrasound waves may capture escaping splatter by pushing it towards a collection point such as, but not limited to, the patient and/or a collection device. To facilitate capturing splatter, the ultrasound waves should be delivered to the area of debridement, which will generally include at least the space between the collection device and the patient. The area of debridement may also include the distance the vibrations induced by the cutting structure travel before they dissipate.

Capturing at least a portion of the splatter by pushing it away from ambient air and towards a collection point, the delivered ultrasound waves may confine the splatter. Splatter pushed towards the patient may be removed with several techniques readily recognizable by those skilled in the art. For instance, the splatter may be removed by aspirating, rinsing, and/or wiping it off the patient. In combination or in the alternative to being pushed towards the patient, the splatter may also be pushed towards a collection device. Any physical structure capable of limiting the propagation of splatter away from wound may be used as a collection device. Accordingly, a simple shield and/or barrier may be used as the collection device. It may be desirable to use a collection device that is capable of holding an amount of the splatter. For instance, a cup and/or pouch may be used as the collection device. Other physical structures in combination with or in addition to those enumerated may be used as a collection device. Regardless of the collection device utilized, at least a portion of the device should be placed over the material to be debrided in front of the point of dissection.

### BRIEF DESCRIPTION OF THE DRAWINGS

The present invention will be shown and described with reference to the drawings of preferred embodiments and clearly understood in its details. Like elements of the various embodiments depicted within the figures are equivalently numbered.

FIG. 1 displays a three-dimensional view of a surgical apparatus that may be used to practice the present invention.

FIG. 2 depicts practice of the present invention with the surgical apparatus depicted in FIG. 1.

### DETAILED DESCRIPTION OF THE INVENTION

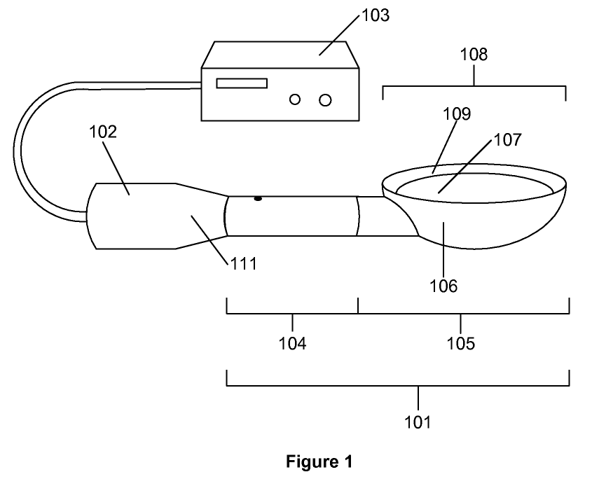

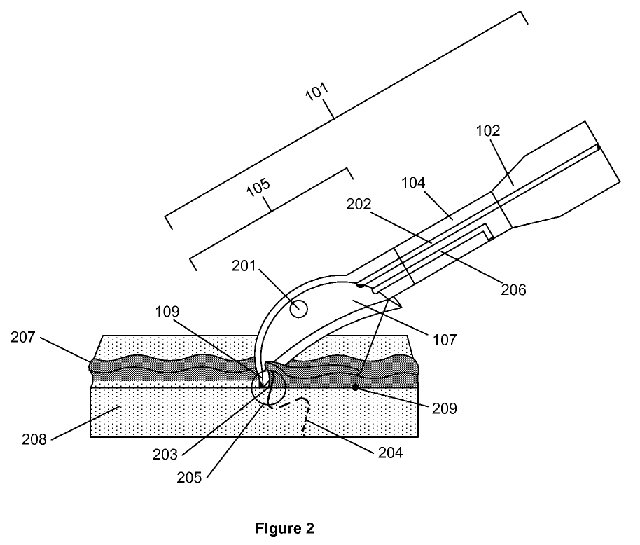

The present invention will be described with reference to the surgical apparatus depicted in FIGS. 1 and 2. It should be appreciated that devices other than the surgical apparatus depicted in FIGS. 1 and 2 may be used to practice the invention. For instance, pending U.S. patent application Ser. Nos. 11/449,220 and 11/857,162, depict devices that may be used to practice the invention, the teachings of which have been previously incorporated by reference.

FIG. 1 displays a three-dimensional view of a surgical apparatus that may be used to the practice the present invention. The advantage of using the surgical apparatus depicted in FIG. 1 is that a single device performs a majority, if not all, of the procedures of the invention. Accordingly, it allows the practitioner practicing the invention to perform several operations with a single motion. The surgical apparatus comprises a surgical substructure 101 attached to an ultrasound transducer 102 driven by an electrical signal produced by generator 103. Surgical substructure 101 is comprised of a shaft 104 and a tip 105. Tip 105, in turn, comprises radial surface 106 and a cavity 107 containing an opening 108. Encircling opening 108 is a sharpened edge 109.

Mechanically coupled to transducer 102 and tip 105, shaft 104 transmits ultrasonic vibrations generated by transducer 102 to tip 105. Accordingly, when driven or otherwise activated by generator 103, transducer 102 induces ultrasonic vibrations within surgical substructure 101. Inducing vibrations in substructure 101 causes ultrasound to be released from the walls of cavity 107 and sharpened edge 109. Transmitting vibrations and releasing ultrasound, surgical substructure 101 may be classified as an ultrasound horn. As such it may beneficial to construct surgical substructure 101 from any material capable of conducting ultrasound vibrations such as, but not limited to, aluminum, stainless steel, titanium, and any combination thereof. It may be preferable to construct surgical substructure 101 from titanium alloy Ti 6Al-4V.

Regardless of the material used to construct surgical substructure 101, generator 103 should be capable of producing an electrical signal of a sufficient voltage to drive transducer 102 to induce substructure 101 to vibrate, preferably in resonance, with the amplitude of the vibrations being between approximately 1 and approximately 250 microns. Surgical substructure 101 may be capable of vibrating approximately in resonance at a frequency between approximately 15 kHz and approximately 5 MHz. Preferably, substructure 101 should be capable of vibrating approximately in resonance at a frequency of approximately 35 kHz.

FIG. 2 depicts practice of the present invention with the surgical apparatus depicted in FIG. 1. To facilitate moistening of the wound, a fluid 202 is delivered to cavity 107 through channel 201, which extends through transducer 102 and shaft 104 before opening into cavity 107. Ultrasound emanating from the walls of cavity 107 atomized and/or breaks fluid 202 into droplets. After atomizing and/or being broken into droplets, fluid 202 is sprayed towards the wound by being forced forwards by ultrasound waves released from the walls of cavity 107. Moistening the wound can also be accomplished with a fluid that is not atomized and/or broken into droplets. Furthermore, the fluid moistening the wound does not need to be applied to the wound with ultrasound waves. Regardless of whether the wound is moistened with an atomized or non-atomized fluid and how the fluid is applied to the wound, a force exerted on the fluid by the ultrasound waves released from the walls of cavity 107 may push at a least a portion of the fluid into the various materials within the wound.

As sharpened edge 109 advances through the wound, fluid 202 in and/or over the wound may flow and/or otherwise come between edge 109 and material within the wound. Fluid between edge 109 and the material within the wound allows sharpened edge 109 to vibrate more freely. In addition to isolating sharpened edge 109 from the material within the wound, fluid 202 in and/or over the wound may act as a conduit carrying vibrations from the edge 109 to, into and/or through material about the point dissection 203.

In addition to in or combination with facilitating the transfer of energy to the material within the wound, the fluid moistening the wound may help to loosen material and/or kill infectious agents to be removed from the wound. The ultrasound waves delivered from the walls of cavity 107 may induce cavitations within fluid 202. In combination or in the alternative, cavitations may be induced within other fluids and/or substances within the wound. Inducing cavitations within the fluids and/or other substances result in the formation of tiny bubbles. Conceptually, this phenomenon is similar to inducing water to boil by applying heat. However, the induction of cavitations by the delivered ultrasound waves is not dependent upon heating the fluid to its boiling point. As such, the induction of cavitations is not dependent upon the transfer of thermal energy.

After spontaneously forming, the cavitations randomly explode and/or collapse. An exploding and/or collapsing cavitation releases energy into the fluid and/or material surrounding it. In combination or in the alternative, the explosion and/or collapse of a cavitation may induce a pressure change within the volume of the fluid surrounding the cavitation. The pressure change and/or energy released may loosen the material to be debrided 207. In the alternative or in combination, infectious agents within the vicinity of the exploding and/or collapsing cavation may be inactivated, killed, weakened, and/or otherwise compromised.

If transducer 102 is a piezoelectric transducer and induces surgical substructure 101 to vibrate approximately in resonance, then the voltage of the electrical signal supplied by generator 103 will largely control the degree to which the fluid 202 moistening the wound is cavitated and/or atomized. At low voltages fluid 202 will be cavitated to a small degree. As the voltage increases, the amount of cavitations within fluid 202 increases. Likewise, as the voltage increases the degree to which fluid 202 is atomized will increase. Regardless, of whether fluid 202 is atomized and/or cavitated, the presence of fluid 202 may facilitate the transfer of energy to the material within the wound by isolating sharpened edge 109 from the material and/or by acting as a conduit carrying vibrations from the edge 109 to, into and/or through material about the point of dissection 203.

When driven or otherwise activated by generator 103, transducer 102 induces ultrasonic vibrations within surgical substructure 101. Mechanically coupled to transducer 102 and tip 105, shaft 104 transmits ultrasonic vibrations generated by transducer 102 to tip 105. Accordingly, ultrasound vibrations generated by transducer 102 are channeled into sharpened structure 109. As the vibrations travel from the wide base to the fine edge of sharpened edge 109, the amplitude of the vibrations increases while the frequency remains constant. Placing the fine edge of sharpened edge 109 in close proximity to the point of dissection 203 induces vibrations 204 within the region 205 about the point of dissection 203. As the vibrations 204 travel through the material within the wound their amplitude decreases. Eventually a distance is reached in which the material is no longer vibrated.

Sharpened structure 109 induces vibrations within region 205 by exposing the material about the point of dissection 203 to concentrated ultrasound vibrations. As the vibrations induced in surgical apparatus 101 by transducer 102 travel into and through the sharpened edge 109 they become concentrated. Accordingly, as the vibrations move towards the fine edge of sharpened edge 109, their amplitude increases while their frequency remains constant. The surgical apparatus depicted in FIG. 2, therefore, induces vibrations in region 205 about the point of dissection 203 by exposing the material about the point of dissection 203 to concentrated ultrasonic vibrations. It should be appreciated that exposing the material about the point of dissection 203 to concentrated ultrasonic vibrations is not necessary to induce vibrations within the material in region 205. Rather, exposing the material about the point of dissection 203 to concentrated ultrasonic vibrations is a consequence of dissecting the material with sharpened edge 109 while the transducer is activated.

The vibrations 204 induced in the material about the point of dissection 203 by exposing the material to the ultrasonic vibrations of sharpened edge 109 travel away from the edge 109 and into the material. As the vibrations travel through the material within the wound, the energy they carry is lost. Losing energy, the amplitude of the vibrations 204 begins to decrease and/or the vibrations dissipate. A portion of the energy lost is absorbed by the adhesion holding the material together and/or to the wound. If the energy absorbed by the adhesion is equal to and/or exceeds the energy required to the break the adhesion, then the material may be separated from the wound and/or fragmented. Accordingly, the energy carried and/or lost by the vibrations 204 induced within the material in the region 205 about the point of dissection 203 selectively separate susceptible adhesions holding material together and/or the wound.

Traveling away from sharpened edge 109, vibrations 204 carry and/or transfer energy to the material within the wound ahead of the edge 109. Consequently, the energy carried by the induced vibrations 204 separate susceptible adhesions ahead of sharpened edge 109. Selective separating susceptible adhesions ahead of sharpened edge 109, the integrity and/or hold of material to be debrided 207 at and/or ahead of the point of dissection 203 becomes weakened and/or lost. With its hold and/or integrity weakened and/or lost, the force that needs to be applied at the point of the dissection 203 by sharpened edge 109 to remove material 207 is reduced. Consequently, material to be debrided 207 may be dissected with a more gentle debridement. The gentle nature of the debridement reduces the chances of removing the material to be kept 208, which may include, but it not limited to, healthy, healing, and/or granulation tissue. In combination or in the alternative, gentle debridement may allow the material 207 to be dissected with reduced levels of tissue and/or cellular damage. Reducing the levels of tissue and/or cellular damage may lead to reduced levels of inflammation and/or pain. Consequently, the vibrations 204 induced at and/or ahead of the point dissection 203 may reduce the burdensome nature of debridement on the patient.

Carrying and/or transferring kinetic energy to the material within the wound, vibrations 204 impart a motion on the removed material. This motion may cause the material removed to fragment and/or atomize. If a sufficient amount of motion is transferred to the material, the fragments and/or droplets may travel away from the wound. Consequently, in addition to selectively separating susceptible adhesion, vibrations 204 may splatter material to be debrided 207 and/or other material such as, but not limited to, fluid 202. Escaping into the ambient air, the splattered material may contaminate other patients and/or the facility.

Placed over the material to be debrided in front of the point of dissection 203, tip 105 limits the propagation of at least some of the splatter into the ambient away. Accordingly, tip 105 functions as a collections device. Containing cavity 107, tip 105 is a capable of holding the splatter it collects. The collected splattered may be removed through channel 206, originating in cavity 107 and running through a portion of shaft 104 before opening within a radial surface of shaft 104. Aspiration may be used to facilitate the removal of the collected splatter through channel 206. In combination or in the alternative, the splatter collected in cavity 107 may be removed by rinsing and/or washing tip. Additional manners for removing splatter from cavity 107, readily recognizable by those skilled in the art, may be used in combination with or in addition to those enumerated.

In addition to collecting splatter, cavity 107 delivers ultrasound waves to the area of debridement. When driven or otherwise activated by generator 103, transducer 102 induces ultrasonic vibrations within surgical substructure 101. When the vibrations reach the walls of cavity 107 they cause the walls to compress forward and rebound backwards. As the walls of cavity 107 compress forward they strike fluid 202 creating a compression within fluid 202. The propagation of this compression forward creates an ultrasound wave carried by fluid 202 that travels in the direction of the compression. If cavity 107 does not contain fluid 202, then the delivered ultrasound waves could be carried by the air, gas, gel, and/or another fluid substance between the walls of cavity 107 and the patient. Because the walls of the cavity 107 face the patient, the ultrasound waves will travel towards the patient. Accordingly, the ultrasound waves delivered from the walls of cavity 107 have trajectories directed towards the patient and away from the ambient air.

Forming a parabola about two axes, the walls of cavity 107 from a paraboloid structured. Due to this paraboloid structure, the ultrasound waves delivered from cavity 107 have trajectories that intersect at focal point 209. It is also possible to utilize delivered ultrasound waves having trajectories that intersect at multiple points to practice the present invention. For example, if the walls of cavity 107 formed a parabola about one axis that was extended about a second axis as to form a vault, then the trajectories of the delivered ultrasound waves would intersect at multiple points. Additional manners and devices for delivering ultrasound waves with intersecting trajectories are possible, and readily recognizable by those skilled in the art.

Regardless of whether the delivered the ultrasound waves have intersecting trajectories, as the waves travel they will exert a forward force about their trajectory on splatter crossing their path. Exerting a forward force on the splatter directed towards the patient, the delivered ultrasound waves will decelerate the splatter's velocity towards the ambient air, pushing it back towards the patient. Pushing the splatter about the direction of its trajectory, the ultrasound waves delivered from the cavity 107 capture at least a portion of the splatter.

Delivering ultrasound waves having trajectories directed towards the patient to capture splatter and collecting splatter within cavity 107, tip 105 reduces and/or eliminates splatter escaping into the ambient air. Consequently, tip 105 reduces and/or eliminates possible contamination by the splatter, thereby reducing the burden on the patient population and/or clinic. Inducing vibrations 204 at and/or ahead of the point of dissection 203 with sharpened edge 109, tip 105 selectively separates susceptible adhesions and/or allows for a more gentle debridement. Consequently, tip 105 may reduce the burdensome nature of debridement on the patient.

Inducing vibrations, dissecting the material to be debrided from the wound, and collecting the resulting, tip 105 provides all the means necessary to practice the present invention. Consequently, practicing the present invention with the surgical apparatus depicted in FIGS. 1 and 2 allows debridement with a single instrument. However, it is not necessary that the present invention be practiced with an apparatus that provides all the necessary means. Accordingly, the vibrating cutting structure, collection device, and/or source of delivered ultrasound waves may be on different instruments. For instance, the present invention may be practiced when the vibrating cutting structure and the source of ultrasound waves are located on two different horns, neither one of which contains the collection device.

Accordingly, though specific embodiments of apparatuses and methods have been illustrated and described herein, it will be appreciated by those of ordinary skill in the art that any arrangement, combination, and/or sequence that is calculated to achieve the same purpose may be substituted for the specific embodiment shown. It is to be understood that the above description is intended to be illustrative and not restrictive. Combinations of the above embodiments and other embodiments as well as combinations and sequences of the above methods will be apparent to individuals possessing skill in the art upon review of the present disclosure.

It should be noted and appreciated that other benefits, mechanisms of action and/or mechanisms of operation, in addition to those listed, may be elicited by methods in accordance with the present invention. The mechanisms of action and mechanism of operation presented herein are strictly theoretical and are not meant in any way to limit the scope of this disclosure and/or the accompanying claims.

It should also be appreciated that elements described with singular articles such as “a”, “an”, and/or “the” and/or otherwise described singularly may be used in plurality. Likewise, it should be appreciated that elements described in plurality may be used singularly.

The scope of the present invention should be determined with reference to the appended claims, along with the full scope of equivalents to which such claims are entitled.

I claim:

1. A method of debriding wounds comprising:
a. moistening a wound;
b. inducing vibrations about a point of dissection with an ultrasonically vibrating cutting structure;
c. dissecting a material to be debrided with the ultrasonically vibrating cutting structure inducing the vibrations;
d. placing a collection device over the material to be debrided in front of the point of dissection; and
e. delivering ultrasound waves to the area of debridement that have trajectories directed towards a collection point.

2. The method according to claim 1 further comprising removing the dissected material from the collection device.

3. The method according to claim 1 characterized by at least a portion of the delivered ultrasound waves having intersecting trajectories.

4. The method according to claim 1 further characterized by the ultrasound waves being delivered from the collection device.

5. The method according to claim 1 further characterized by the ultrasound waves being carried by the fluid used to moisten the wound.

6. The method according to claim 1 further comprising inducing cavitations in a fluid within the wound.

7. The method according to claim 1 characterized by the delivered ultrasound waves having a frequency between approximately 15 kHz and approximately 5 MHz.

8. The method according to claim 1 characterized by the delivered ultrasound waves having an amplitude between 1 and 250 microns.

9. The method according to claim 1 characterized by the cutting structure inducing vibration about the point of dissection vibrating at a frequency between approximately 15 kHz and approximately 5 MHz.

10. The method according to claim 1 characterized by the cutting structure inducing vibration about the point of dissection vibrating at an amplitude between approximately 1 and approximately 250 microns.

11. A method of debriding wounds comprising:
a. moistening a wound;
b. inducing vibrations about a point of dissection with an ultrasonically vibrating cutting structure;
c. dissecting a material to be debrided with the ultrasonically vibrating cutting structure inducing the vibrations;
d. placing a collection device over the material to be debrided in front of the point of dissection; and
e. delivering ultrasound waves to the area of debridement that have intersecting trajectories towards a collection point.

12. The method according to claim 11 further comprising removing the dissected material from the collection device.

13. The method according to claim 11 further characterized by the ultrasound waves being delivered from the collection device.

14. The method according to claim 11 further characterized by the ultrasound waves being carried by the fluid used to moisten the wound.

15. The method according to claim 11 further comprising inducing cavitations in a fluid within the wound.

16. The method according to claim 11 characterized by the delivered ultrasound waves having a frequency between approximately 15 kHz and approximately 5 MHz.

17. The method according to claim 11 characterized by the delivered ultrasound waves having an amplitude between 11 and 250 microns.

18. The method according to claim 11 characterized by the cutting structure inducing vibration about the point of dissection vibrating at a frequency between approximately 15 kHz and approximately 5 MHz.

19. The method according to claim 11 characterized by the cutting structure inducing vibration about the point of dissection vibrating at an amplitude between approximately 1 and approximately 250 microns.

20. A method of debriding wounds comprising:
a. moistening a wound;
b. inducing vibrations about a point of dissection with an ultrasonically vibrating cutting structure;
c. dissecting a material to be debrided with the ultrasonically vibrating cutting structure inducing the vibrations;
d. placing a collection device over the material to be debrided in front of the point of dissection; and
e. delivering ultrasound waves from the collection device to the area of debridement that have trajectories directed towards a point of collection.

21. The method according to claim 20 further comprising removing the dissected material from the collection device.

22. The method according to claim 20 characterized by at least a portion of the delivered ultrasound waves having intersecting trajectories.

23. The method according to claim 20 further characterized by the ultrasound waves being carried by the fluid used to moisten the wound.

24. The method according to claim 20 further comprising inducing cavitations in a fluid within the wound.

25. The method according to claim 20 characterized by the delivered ultrasound waves having a frequency between approximately 15 kHz and approximately 5 MHz.

26. The method according to claim 20 characterized by the delivered ultrasound waves having an amplitude between 1 and 250 microns.

27. The method according to claim 20 characterized by the cutting structure inducing vibration about the point of dissection vibrating at a frequency between approximately 15 kHz and approximately 5 MHz.

28. The method according to claim 20 characterized by the cutting structure inducing vibration about the point of dissection vibrating at an amplitude between approximately 1 and approximately 250 microns.
